(19) United States
(12) Patent Application Publication
(10) Pub. No.: US 2021/0244911 A1
(43) Pub. Date: Aug. 12, 2021

(54) PERCUTANEOUS PULMONARY ARTERY DRAINAGE DEVICE

(71) Applicants: University of Kentucky Research Foundation, Lexington, KY (US); W-Z Biotech, LLC, Lexington, KY (US)

(72) Inventors: Dongfang Wang, Lexington, KY (US); Joseph B. Zwischenberger, Lexington, KY (US); Guangfeng Zhao, Shenzhen (CN); Cherry Ballard-Croft, Lexington, KY (US)

(21) Appl. No.: 17/232,594

(22) Filed: Apr. 16, 2021

Related U.S. Application Data
(63) Continuation-in-part of application No. PCT/US2019/056767, filed on Oct. 17, 2019.
(60) Provisional application No. 62/746,950, filed on Oct. 17, 2018.

(51) Int. Cl. A61M 25/00

(52) U.S. Cl. CPC A61M 25/003; A61M 25/0074; A61M 2025/0031; A61M 2210/125; A61M 2205/0216; A61M 2205/3331; A61M 2210/127; A61M 2025/0002

(57) ABSTRACT

A multiple lumen device for percutaneous left ventricular unloading during venoarterial extracorporeal membrane oxygenation comprising: an expandable cage, an outer catheter, and an inner catheter, wherein a proximal end of the expandable cage is attached to the outer catheter and a distal end of the expandable cage is attached to the inner catheter, such that when the proximal end of the inner catheter is retracted, the cage moves from a compressed configuration to an expanded configuration is described. Also provided are methods for causing the blood of a subject to flow in a retrograde manner using a multiple lumen device.

### RELATED APPLICATIONS

This application claims priority from International Patent Application No. PCT/US19/56767 filed on Oct. 17, 2019 and U.S. Provisional Patent Application No. 62/746,950 filed on Oct. 17, 2018, the entire disclosures of which are incorporated herein by this reference.

### GOVERNMENT INTEREST

This invention was made with government support under grant number SBIR R44HL 129490/3-200001236 awarded by the National Institutes of Health. The government has certain rights in the invention.

### TECHNICAL FIELD

The present disclosure is directed to devices for catheterization of the heart. More specifically, the disclosure is directed to devices for percutaneous left ventricular unloading during venoarterial extracorporeal membrane oxygenation.

### INTRODUCTION

Cardiogenic shock (CS) is a serious condition of reduced cardiac output (CO) with end organ hypoperfusion. CS is mainly caused by acute myocardial infarction, but it can also originate from heart valve disease, myocarditis, arrhythmias, and drug toxicity. Even with recent advances in treatment, CS mortality is still as high as 40-50%¹⁻³. In severe CS, two critical pathophysiological mechanisms lead to patient death: 1) Low CO causes end organ hypoperfusion, resulting in multi-organ failure; 2) Significantly elevated left ventricle (LV) preload increases LV wall stress, exacerbating myocardial injury and preventing recovery. Mechanical circulatory support (MCS) provides sufficient end organ perfusion and is expected to unload the LV, bridging the CS patient to recovery, further treatment, long-term LV assist device (LVAD), or heart transplantation. Venoarterial extracorporeal membrane oxygenation (VA ECMO) is increasingly used as MCS for severe CS⁴ due to its wide availability, total circulatory support, cost effectiveness, and minimally invasive access. VA ECMO pumps up to total CO to normalize end-organ perfusion, reducing the risk of multi-organ failure.

However, VA ECMO is unable to unload the LV in severe CS patients, and more than 50% of these patients develop LV distension⁵, further damaging the myocardium and preventing myocardial recovery. In VA ECMO, a small amount of venous return still goes to the left heart, but the compromised LV is unable to pump this blood out against systemic pressure, resulting in LV distension, increased wall tension, and decreased endocardial blood flow. Current LV unloading techniques include off-label use of a cannula for left atrium (LA)/pulmonary artery (PA) drainage, atrial septostomy, or installation of an additional blood pump, which are too invasive, require highly specialized personnel, or need very specialized/expensive equipment. Currently, there is no minimally invasive device specifically designed for unloading the LV during VA ECMO

Mechanical Circulatory Support (MCS) is required to correct pathophysiology of severe CS by: 1) Providing sufficient systemic blood flow for end organ perfusion to prevent multi-organ failure and 2) Decreasing LV preload to unload left heart, preventing further myocardial injury and allowing recovery. MCS maintains systemic circulation and unloads the LV, bridging severe CS patient for: 1) Further advanced treatment to address the cause of CS; 2) Myocardial/end organ recovery; 3) Long-term LVAD/total artificial heart; 4) Heart transplantation.

Currently available percutaneous short-term MCS includes Intra-aortic balloon pump (IABP), TandemHeart, and Impella 2.5. IABP is the most frequently used short-term MCS for CS due to its availability and rapid implantation⁶. However, IABP has very limited capacity for LV support without a significant impact on CS⁷.

Percutaneous Impella® 2.5 and Tandemheart™ have proven hemodynamic benefit, and their application has increased in the last decade^{6,8}. However, percutaneous Impella 2.5 and Tandem Heart only provide partial circulatory support with no survival improvement⁸⁻¹⁰.

Non-percutaneous short-term MCS (Thoratec PVAD, ABS/BVS 5000, Impella® 5.0 and CentriMag) require major open chest surgery for installation, with high cost, limited availability, high complication rate, and low utilization⁶.

In summary, the available percutaneous MCS devices have limited circulatory support capacity, which is not enough to stabilize circulation in severe CS. Non-percutaneous MCS devices supply up to total cardiac support, but require invasive open chest surgery and specialized expertise for installation.

By contrast, peripheral venoarterial extracorporeal membrane oxygenation (VA ECMO) provides up to total circulatory support without major open chest surgery. VA ECMO is the fastest way to stabilize a patient in acute CS and prevent end organ failure¹¹. VA ECMO utilization has sharply increased in severe CS^{4,6,12} due to its convenience, cost effectiveness, wide availability, and minimally invasiveness. However, the in-hospital mortality is still high in severe CS patients on VA ECMO (>50%)⁴ VA ECMO provides total circulatory support to prevent multi-organ failure and extend patient life in the short-term. However, VA ECMO is unable to unload the LV in severe CS patients, and more than 50% of these patients develop LV distension⁵, further damaging the myocardium and preventing myocardial recovery.

In VA ECMO, a small amount of venous return goes to left heart, but the compromised LV is often unable to pump this blood out against systemic pressure, resulting in LV distension. The consequences of LV distension are: 1) Increased LV wall stress with endocardial ischemia further damages myocardium and prevents recovery. 2) Significantly increased pulmonary capillary pressure to the level of LAP (>20 mmHg), causes lung edema and compromises lung function. LV distention is very common in VA ECMO for CS, with an incidence as high as 50%⁵. The actual LV distension incidence may be even higher, due to the difficulty of LAP measurement during ECMO.

To reverse this pathophysiology, LV unloading is required to prevent further myocardial damage, allowing recovery and improving severe CS outcomes^{13,14}. Non-percutaneous, short-term MCS, or direct LA/PA drainage cannulation can achieve total LV unloading in severe CS supported by VA ECMO. However, open chest surgery, expensive/complex equipment, and specific expertise is required, which is unsuitable for widespread application.

Percutaneous techniques have been used for LV unloading in VA ECMO supported severe CS patients, but there are significant disadvantages that limit their use. The following are illustrative of limitations in present devices: 1) IABP is simple and available in all ECMO programs, but has very limited LV unloading capacity. 2) Impella 2.5, Tandemheart LA cannula, and transeptal atrial septostomy are complicated, expensive, and require specialized expertise, which is not commonly available. 3) Off-label supplies (such as PA cannula drainage, Transeptal cannula) have limited availability of the desired cannula size/length. Even introducer guide sheaths, which are not designed for blood flow, are occasionally used for LV unloading²¹, with limited success. 4) Most of the above techniques (PA cannula, LA cannula, Impella 2.5) need real time manual pump adjustments to optimize LV unloading performance because of the negative pressure required for blood withdrawal. Therefore, an additional method for monitoring LV unloading status is required to adjust this negative pressure for desired blood flow²⁰. Precise manual adjustments are required because too much blood withdrawal by a PA cannula can easily collapse and completely obstruct the PA, disabling LV unloading while too little withdrawal leads to ongoing LV distension. 5) None of the above techniques have an integrated method to monitor/evaluate the LV unloading status, which is highly desired for optimal LV unloading.

The concept of unloading the LV via PA valve regurgitation was patented 20 years ago by Theodor Kolobow. He used a cardiopulmonary bypass sheep model with unloading by a directly inserted PA catheter with an attached fixed spring/helical coil.²⁷⁻³⁰. His technology was never developed or commercialized. One embodiment of the present invention was specifically designed with a collapsible/expandable metal cage to relieve LV distension in severe CS during VA ECMO. An embodiment of the present invention is easy to insert percutaneously and addresses a specific current clinical problem due to growing utilization of VA ECMO for CS resuscitation.

An embodiment of the present invention is a percutaneous PA drainage device (pPADD) that keeps the PA/tricuspid valves open, allowing retrograde blood flow from the PA toward the right atrium for ECMO drainage. This results in a lower PA pressure (PAP) which not only decreases blood flow from the PA to LA, but also enables retrograde blood flow from the LA to the PA to unload the LV.

A further embodiment of the present invention is a percutaneous PA drainage device that is small enough for neonates and large enough for large adults.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of features.

One embodiment of the present invention includes a multiple lumen device, comprising: (a) an expandable cage having a proximal end and a distal end; (b) an outer catheter comprising a first lumen and having a proximal end and a distal end; (c) an inner catheter comprising a second lumen and a third lumen, and having a proximal end and a distal end, the inner catheter extending through the outer catheter and the expandable cage, and extending beyond the distal end of the expandable cage, wherein the proximal end of the expandable cage is attached to the outer catheter and the distal end of the expandable cage is attached to the inner catheter, such that when the proximal end of the inner catheter is retracted, the cage moves from a compressed configuration to an expanded configuration.

In further embodiments of the present invention, the device includes a pressure monitor in fluid communication with the outer catheter for measuring pressure in the right atrium (RA). In some embodiments, the device may include an inflatable balloon at the distal tip of and in fluid communication with the second and third lumens of the inner catheter. In other embodiments of the present invention, the device further comprising a syringe in fluid communication with the second lumen of the inner catheter for inflating the balloon. In some embodiments of the present invention, the device further comprises a pressure monitor in fluid communication with the third lumen of the inner catheter for measuring for measuring pressure in the pulmonary artery (PAP) and pulmonary artery wedge pressure (PCWP).

In some embodiments of the present invention, the wire cage is comprised of super-elastic nitinol wire, stainless steel wire, super elastic polymer, or combinations thereof. In other embodiments, at least two surgical grade threads are placed around the cage at equal distance from each other. In certain embodiments of the present invention, the surgical grade threads are comprised of polyester, polypropylene, nylon, or combinations thereof. In other embodiments of the present invention, at least one of the outer catheter or the inner catheter is coated with polytetrafluoroethylene. In other embodiments of the present invention, the device is comprised of polyurethane, PVC, silicone, PFTE, polyisoprene, nitrile, or combinations thereof. In some embodiments, the cage has a maximal expanded diameter from 12 mm to 15 mm.

Another embodiment of the present invention is a method for causing blood to flow in a retrograde manner from the pulmonary artery into the right atrium, comprising: (a) selecting a multiple lumen device comprising: (i) an expandable cage having a proximal end and a distal end; (ii) an outer catheter comprising a first lumen and having a proximal end and a distal end; (iii) an inner catheter comprising a second lumen and a third lumen, and having a proximal end and a distal end, the inner catheter extending through the outer catheter and the expandable cage, and extending beyond the distal end of the expandable cage, wherein the proximal end of the expandable cage is attached to the outer catheter and the distal end of the expandable cage is attached to the inner catheter, such that when the proximal end of the inner catheter is retracted, the cage moves from a compressed configuration to an expanded configuration; (b) percutaneously inserting the multiple lumen device into a blood vessel that leads to the heart; (c) continuing to insert the multiple lumen device until the distal end of the cage is through the pulmonary valve and into the pulmonary artery; (d) retracting the inner catheter until the cage is in an expanded configuration; (e) allowing the desired amount of blood to flow in a retrograde manner from the pulmonary artery into the right atrium; (f) extending the inner catheter until the cage is in a compressed configuration; and (g) removing the multiple lumen device from the blood vessel.

Additional features and advantages of the systems and methods of the present disclosure will become evident to those of ordinary skill in the art after a study of the description, figures, and non-limiting examples in this document.

### BRIEF DESCRIPTION OF THE DRAWINGS

FIG. 1 illustrates an embodiment of a percutaneous pulmonary artery drainage device (pPADD). The pPADD causes tricuspid/PA valve regurgitation with pulmonary artery-Right atrium retrograde blood flow for left ventricular unloading.

FIGS. 2A-B illustrate the prior art. FIG. 2A shows product placement in the right jugular vein and inferior vena cava. FIG. 2B shows product placement in the heart showing the critical nature of placing the device with correct orientation of the return jet towards the tricuspid valve.

FIG. 3 illustrates one embodiment of the present invention containing an inflatable balloon.

FIGS. 4A-4B illustrate an exemplary embodiment of an expandable metal cage on a pPADD and an inflatable balloon. FIG. 4A. shows the cage collapsed. FIG. 4B. shows the cage expanded.

FIG. 5 illustrates an exemplary pPADD that is in a working position, where the expandable cage is in expanded position inside a heart.

FIGS. 6A-6B illustrate an exemplary embodiment of the device of the present invention.

FIGS. 7A-7C illustrate another exemplary embodiment of the device of the present invention.

### DETAILED DESCRIPTION OF THE EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

All references to singular characteristics or limitations of the present disclosure shall include the corresponding plural characteristic(s) or limitation(s) and vice versa, unless otherwise specified or clearly implied to the contrary by the context in which the reference is made.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. For example, reference to “a device” includes a plurality of devices, and so forth.

Unless otherwise indicated, all numbers expressing quantities, properties, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, length, width, weight, time, volume, concentration or percentage is meant to encompass variations of in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “approximately” are used interchangeably and carry the same meaning.

While the following terms used herein are believed to be well understood by one of ordinary skill in the art, definitions are set forth to facilitate explanation of the presently-disclosed subject matter.

The terms “subject” or “subject in need thereof” refer to a target in need of intervention, wherein the subject optionally displays symptoms related to a particular disease, pathological condition, disorder, or the like. The subject of the herein disclosed methods can be a human, non-human primate, horse, pig, dog, sheep, goat, or cow. The term “subject” does not denote a particular age or sex. Thus, adult and newborn subjects, as well as fetuses, whether male or female, are intended to be covered. A “subject” refers to a subject afflicted with a disease or disorder. The term “subject” includes human and veterinary subjects.

The terms “treatment” or “treating” refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder and/or resulting symptoms of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The present invention relates to a device the left ventricle to be unloaded during venoarterial extracorporeal membrane oxygenation (ECMO) in a minimally-evasive manner. Accordingly, such a device can be useful, for example, to facilitate myocardial recovery after cardiogenic shock.

Uniquely, the device keeps both the pulmonary artery valve and tricuspid valves open simultaneously by threading through both the pulmonary artery valve and tricuspid valves, and when the wire cage is expanded, allowing blood to flow in a retrograde manner from the PA toward the right atrium for improved ECMO drainage. The device therefore creates lower pulmonary artery pressure, decreases blood flow from pulmonary artery to left atrium, and allows retrograde blood flow from the left atrium to the pulmonary artery to unload the left ventricle. Because the operator has fine control over the expansion of the metal cage in both valves, it allows for the appropriate amount of ECMO drainage can occur.

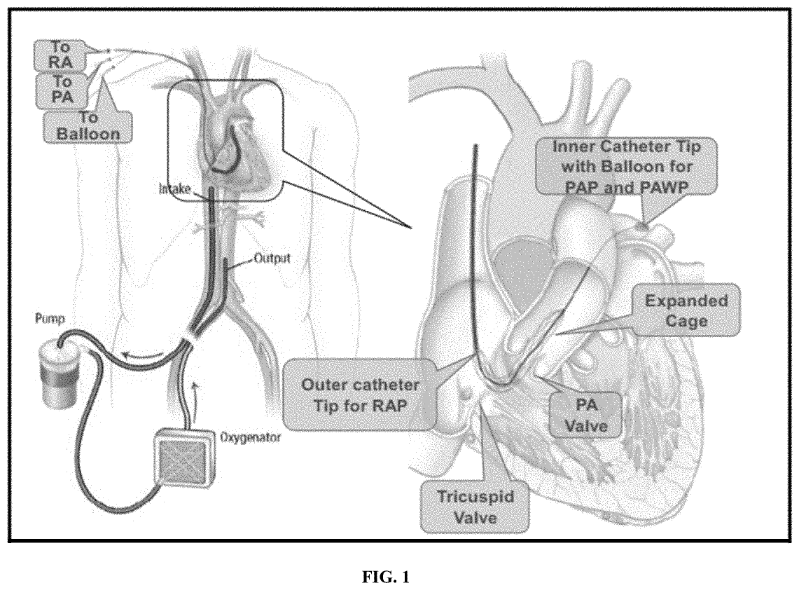

In some embodiments, as illustrated in FIGS. 1, 3, 4A-B, 5, and 6A-B the presently-disclosed subject matter includes a device for allowing unloading of the LV during VA ECMO.

Referring to FIG. 1, in one embodiment, the device includes a 7 FR triple lumen catheter with a cylindrical memory alloy wire cage located near the distal end of the catheter, the catheter having features arranged and disposed to collapse and expand the wire cage for easy percutaneous insertion from jugular or femoral vein. For example, in another embodiment, the wire cage is expanded from about 2.5 mm to about 15 mm to create valve regurgitation. In a further embodiment, the wire cage is expanded by withdrawal of the 5 FR inner catheter externally towards the proximal end of the catheter.

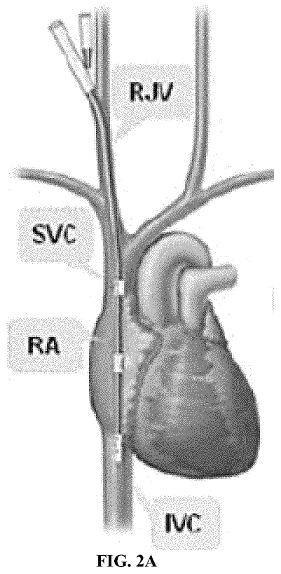

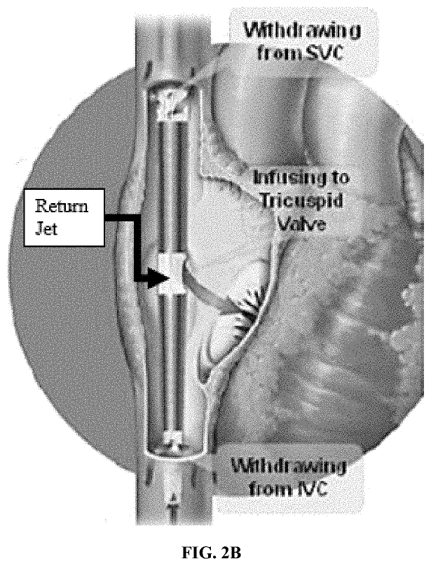

FIGS. 2A and 2B, describe a device of the prior art. The prior art devices are large in size compared to embodiments of the present invention. FIG. 2A shows an overview of the heart and placement of the prior art device within the jugular vein. Therefore, the prior art devices are limited to being used with individuals with large jugular veins and predispose patients to hemolysis at high flow rates. Furthermore, proper cannula placement is critical and depends on the correct orientation of the return jet otherwise the return jet will not be directed towards the tricuspid valve (FIG. 2B).

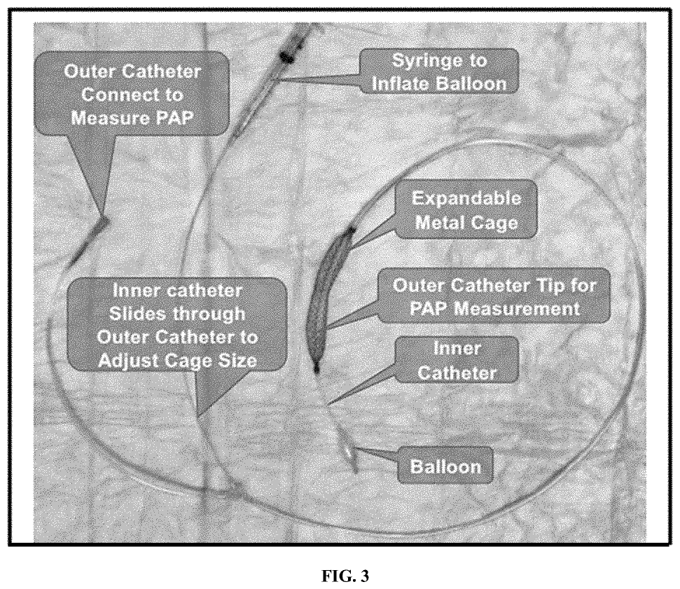

Referring to FIG. 3, in some embodiments, the device includes an inflatable balloon on the distal tip of the inner catheter.

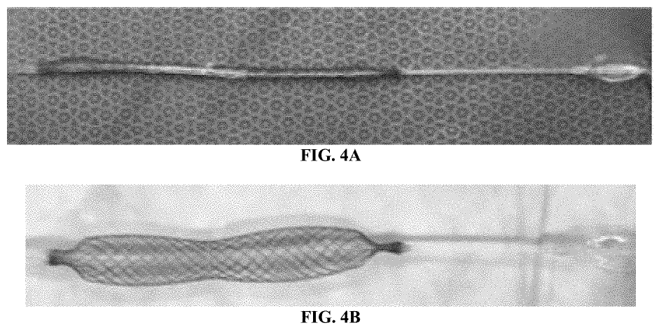

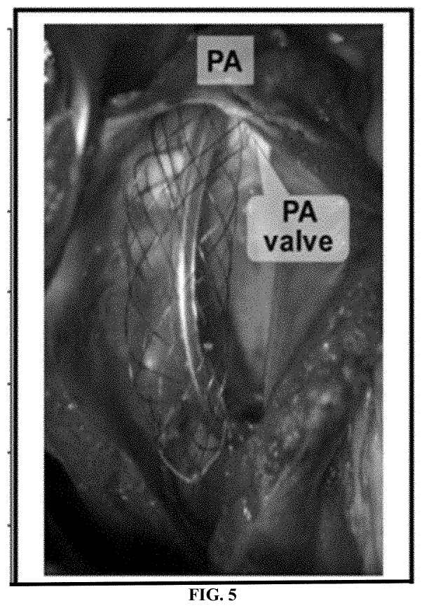

Referring to FIGS. 4A-B, 5, and 6A-B, in some embodiments, the device comprises a coaxial triple lumen catheter and an expandable cage, the catheter further comprised of an inner and outer catheter wherein the inner catheter is inside the outer catheter and sealed through a 3-way connection near the proximal end of the catheter and extends beyond the distal end of the outer catheter by approximately 250 mm. In a further embodiment, the inner catheter has 2 lumens, wherein one lumen is for inflation of a balloon on the distal tip of the inner catheter and the other lumen is for a guide wire and PA pressure measurement device. In another embodiment, the connection between the inner and outer catheter is sealed by a silicone membrane sleeve having features arranged and disposed to allow for the inner catheter to move proximally and distally approximately 3 cm to expand and collapse the wire cage. In other embodiments, the wire cage is comprised of approximately 0.1 mm woven super-elastic nitinol wire. In further embodiments the total length of the wire cage is approximately 90 mm when the wire cage is collapsed. In some embodiments, the proximal end of the wire cage is smoothly molded onto the proximal end of the outer catheter end without adding additional width or length to the outer catheter and the distal end of the wire cage is smoothly molded near the distal end inner catheter. In other embodiments, at least 2 surgical grade polyester threads about 0.01 mm in diameter are placed around the wire cage at equal distance from each other in such a manner to restrain the maximal expanded diameter to about 15 mm. In another embodiment, the wire cage has a maximal expanded length of 60 mm.

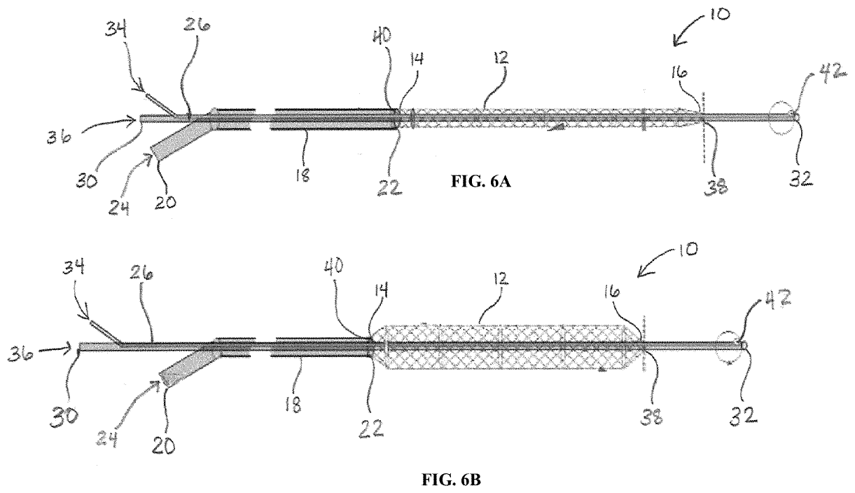

Referring to FIGS. 6A-B, an exemplary embodiment of the device (10) of the present invention includes an expandable cage (12) having a proximal end (14) and a distal end (16), an outer catheter (18) having a proximal end (20) and a distal end (22), and an inner catheter (26) having a proximal end (30) and a distal end (32). The outer catheter includes a first lumen (24). The inner catheter (26) includes a second lumen (34) and a third lumen (36). The inner catheter (26) extends through the outer catheter (18) and the expandable cage (12), such that the distal end (32) of the inner catheter (26) extends beyond the distal end (16) of the expandable cage (12). The proximal end (14) of the expandable cage (12) is attached (40) to the outer catheter and the distal end (16) of the expandable cage (12) is attached to the inner catheter (26), such that when the proximal end (30) of the inner catheter (30) is retracted (FIG. 6B), the cage moves from a compressed/collapsed configuration (FIG. 6A) to an expanded configuration (FIG. 6B).

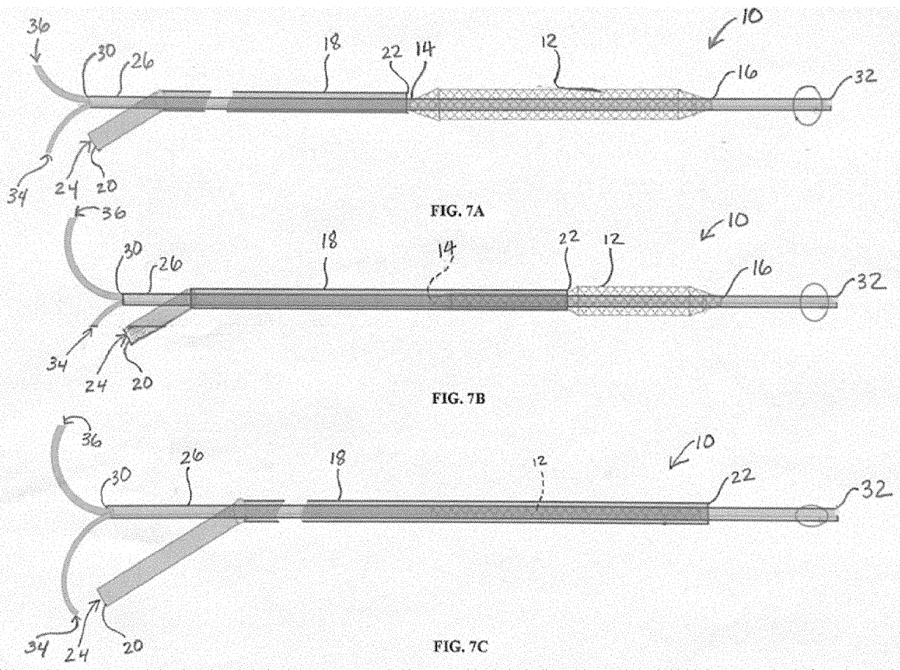

Referring to FIGS. 7A-B, an exemplary embodiment of the device (10) of the present invention includes an expandable cage (12) having a proximal end (14) and a distal end (16), an outer catheter (18) having a proximal end (20) and a distal end (22), and an inner catheter (26) having a proximal end (30) and a distal end (32). The outer catheter includes a first lumen (24). The inner catheter (26) includes a second lumen (34) and a third lumen (36). The inner catheter (26) extends through the outer catheter (18) and the expandable cage (12), such that the distal end (32) of the inner catheter (26) extends beyond the distal end (16) of the expandable cage (12). At least one of the proximal end (14) and the distal end (16) of the expandable cage (12) is attached to the inner catheter (26), such that when the proximal end (30) of the inner catheter (30) is retracted, the cage (12) moves from outside (FIG. 7A) the outer catheter (26), to become partially folded within (FIG. 7B) the outer catheter (26), and can become fully folded within (FIG. 7C) the outer catheter (26). As depicted, the proximal end (14) of the expandable cage (12) can be tapered to facilitate entry into the outer catheter (26) to become folded/collapsed therein.

In this manner, and with continued reference to FIGS. 7A-7C, the expandable cage can be folded/collapsed within the outer catheter during insertion and withdrawal, which can contribute to the ease of insertion and withdrawal, and to the prevention of potential damage to a vessel or tissue from movement of the expandable cage during insertion and withdrawal. In some cases, the exemplary embodiment can be employed as follows. With regard to insertion/deployment, after confirmation of proper positioning of the folded cage inside the tricuspid and pulmonary artery (PA) valves, e.g., with imaging, the inner catheter can be held while the outer catheter can be retracted. This allows the cage to extend out of the distal end of the outer catheter for expansion, creating tricuspid/PA valve regurgitation. With regard to withdrawal, the expanded cage can also be folded/collapsed inside the outer catheter lumen by retracting the inner catheter for easy and safe removal.

According to one or more of the embodiments disclosed herein, the device is formed by polyurethane (PU) dip molding, synthetic polyisoprene dip molding, silicone dip molding, polytetrafluoropolymer molding, polyvinyl chloride molding, or nitrile dip molding.

According to other embodiments disclosed herein, the outer surface of the inner catheter and/or the inner surface of the outer catheter may be coated with polytetrafluoroethylene to decrease the sliding friction for easy metal cage expansion.

In another embodiment disclosed herein, the cage has a maximal expanded size of about 12 mm.

The catheter and connecter is formed from any suitable material for insertion and/or fixation within an individual's body. Accordingly, as will be appreciated by those skilled in the art, the material of the device may vary. Suitable materials for the device include, but are not limited to, polyurethane, PVC, silicone, PTFE, polyisoprene, nitrile, or a combination thereof.

The seal for the connection between the inner and outer catheters may be sealed by a number of suitable materials, including but not limited to silicone membrane sleeve, PVC, PTFE, polyisoprene, nitrile, or combination thereof.

The wire cage is woven to provide strength and elasticity. Accordingly it will be appreciated by those skilled in the art, the material of the wire cage may vary. Suitable materials for the wire cage include, but are not limited to, super-elastic nitinol wire, stainless steel wire, super elastic polymer, or combinations thereof.

At least 2 approximately 0.1 mm surgical grade threads are placed around the wire cage to maintain an even cylindrical shape and restrain the maximal diameter of the expanded cage to approximately 15 mm. Suitable materials for the surgical grade threads include, but are not limited to, polyester, polypropylene, nylon or combinations thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference

It will be understood that various details of the presently disclosed subject matter can be changed without departing from the scope of the subject matter disclosed herein. Furthermore, the foregoing description is for the purpose of illustration only, and not for the purpose of limitation.

### REFERENCES

1. Jeger, R. V., et al. Ten-year trends in the incidence and treatment of cardiogenic shock. Annals of internal medicine 149, 618-626 (2008).
2. Hochman, J. S., et al. Cardiogenic shock complicating acute myocardial infarction—etiologies, management and outcome: a report from the SHOCK Trial Registry. Should we emergently revascularize Occluded Coronaries for cardiogenic shock? J. Am. Coll. Cardiol. 36, 1063 (2000).
3. Goldberg, R. J., Spencer, F. A., Gore, J. M., Lessard, D. & Yarzebski, J. Thirty-year trends (1975 to 2005) in the magnitude of, management of, and hospital death rates associated with cardiogenic shock in patients with acute myocardial infarction: a population-based perspective. Circulation 119, 1211-1219 (2009).
4. Thiagaraj an, R. R., et al. Extracorporeal Life Support Organization Registry International Report 2016. Asaio j 63, 60-67 (2017).
5. Truby, L. K., et al. Incidence and Implications of Left Ventricular Distention During Venoarterial Extracorporeal Membrane Oxygenation Support. ASAIO J 63, 257-265 (2017).
6. Stretch, R., Sauer, C. M., Yuh, D. D. & Bonde, P. National trends in the utilization of short-term mechanical circulatory support: incidence, outcomes, and cost analysis. J Am Coll Cardiol 64, 1407-1415 (2014).
7. Thiele, H., et al. Intraaortic balloon support for myocardial infarction with cardiogenic shock. N Engl J Med 367, 1287-1296 (2012).
8. Miller, P. E., Solomon, M. A. & McAreavey, D. Advanced Percutaneous Mechanical Circulatory Support Devices for Cardiogenic Shock. Crit Care Med 45, 1922-1929 (2017).
9. Kar, B., Gregoric, I. D., Basra, S. S., Idelchik, G. M. & Loyalka, P. The percutaneous ventricular assist device in severe refractory cardiogenic shock. J Am Coll Cardiol 57, 688-696 (2011).
10. Ouweneel, D. M., Eriksen, E., Seyfarth, M. & Henriques, J. P. Percutaneous Mechanical Circulatory Support Versus Intra-Aortic Balloon Pump for Treating Cardiogenic Shock: Meta-Analysis. J Am Coll Cardiol 69, 358-360 (2017).
11. Shishehbor, M. H., et al. Cardiogenic shock: From ECMO to Impella and beyond. Cleve Clin J Med 84, 287-295 (2017).
12. Shah, M., et al. Trends in mechanical circulatory support use and hospital mortality among patients with acute myocardial infarction and non-infarction related cardiogenic shock in the United States. Clinical research in cardiology: official journal of the German Cardiac Society (2017).
13. Schmack, B., et al. Extracorporeal life support with left ventricular decompression-improved survival in severe cardiogenic shock: results from a retrospective study. Peer J 5, e3813 (2017).
14. Pappalardo, F., et al. Concomitant implantation of Impella® on top of veno-arterial extracorporeal membrane oxygenation may improve survival of patients with cardiogenic shock. Eur J Heart Fail 19, 404-412 (2017).
15. Beurtheret, S., Mordant, P., Pavie, A. & Leprince, P. Impella and extracorporeal membrane oxygenation: a demanding combination. ASAIO J 58, 291-293 (2012).
16. Barbone, A., Malvindi, P. G., Ferrara, P. & Tarelli, G. Left ventricle unloading by percutaneous pigtail during extracorporeal membrane oxygenation. Interact Cardiovasc Thorac Surg 13, 293-295 (2011).
17. Hong, T. H., et al. Initial Experience of Transaortic Catheter Venting in Patients with Venoarterial Extracorporeal Membrane Oxygenation for Cardiogenic Shock. ASAIO J 62, 117-122 (2016).
18. Kotani, Y., et al. Left atrial decompression during venoarterial extracorporeal membrane oxygenation for left ventricular failure in children: current strategy and clinical outcomes. Artif Organs 37, 29-36 (2013).
19. Hacking, D. F., et al. Elective decompression of the left ventricle in pediatric patients may reduce the duration of venoarterial extracorporeal membrane oxygenation. Artif Organs 39, 319-326 (2015).
20. Bernhardt, A. M., et al. Percutaneous left atrial unloading to prevent pulmonary oedema and to facilitate ventricular recovery under extracorporeal membrane oxygenation therapy. Interact Cardiovasc Thorac Surg (2017).
21. Aiyagari, R. M., Rocchini, A. P., Remenapp, R. T. & Graziano, J. N. Decompression of the left atrium during extracorporeal membrane oxygenation using a transseptal cannula incorporated into the circuit. Crit Care Med 34, 2603-2606 (2006).
22. Baruteau, A. E., et al. Percutaneous balloon atrial septostomy on top of venoarterial extracorporeal membrane oxygenation results in safe and effective left heart decompression. European heart journal. Acute cardiovascular care (2016).
23. Lin, Y. N., et al. Atrial Septostomy for Left Atrial Decompression During Extracorporeal Membrane Oxygenation by Inoue Balloon Catheter. Circ J 81, 1419-1423 (2017).
24. von Segesser, L. K., Kwang, K., Tozzi, P., Horisberger, J. & Dembitsky, W. A simple way to decompress the left ventricle during venoarterial bypass. Thorac Cardiovasc Surg 56, 337-341 (2008).
25. Fouilloux, V., Lebrun, L., Mace, L. & Kreitmann, B. Extracorporeal membranous oxygenation and left atrial decompression: a fast and minimally invasive approach. Ann Thorac Surg 91, 1996-1997 (2011).
26. Avalli, L., et al. Percutaneous left-heart decompression during extracorporeal membrane oxygenation: an alternative to surgical and transeptal venting in adult patients. ASAIO J 57, 38-40 (2011).
27. Foti, G., et al. Cardiopulmonary bypass through peripheral cannulation with percutaneous decompression of the left heart in a model of severe myocardial failure. Asaio j 43, 927-931 (1997).
28. Kolobow, T., Rossi, F., Borelli, M. & Foti, G. Long-term closed chest partial and total cardiopulmonary bypass by peripheral cannulation for severe right and/or left ventricular failure, including ventricular fibrillation. The use of a percutaneous spring in the pulmonary artery position to decompress the left heart. ASAIO Trans 34, 485-489 (1988).
29. Rossi, F., Kolobow, T., Foti, G., Borelli, M. & Mandava, S. Long-term cardiopulmonary bypass by peripheral cannulation in a model of total heart failure. The decompression of the left heart through a percutaneous helical spring positioned within the lumen of the tricuspid and pulmonary artery valves. J. Thorac. Cardiovasc. Surg. 100, 914 (1990).
30. Kolobow, T. Method for improved use of heart/lung machine Vol. U.S. Pat. No. 4,889,137 (The United States Of America As Represented By The Department Of Health And Human Services, 1989).
31. Wang, D. & Zwischenberger, J. B. Single expandable double lumen cannula assembly for veno-venous ECMO in USPTO (ed. USPTO) (The University of Texas System USA, 2009).
32. Evans, S. Patients Up and About With Portable, Artificial Lung. (ABC News, Apr. 2, 2009).
33. Chimot, L., et al. Avalon(c) bicaval dual-lumen cannula for venovenous extracorporeal membrane oxygenation: survey of cannula use in France. Asaio j 59, 157-161 (2013).
34. Reeb, J., Falcoz, P. E., Santelmo, N. & Massard, G. Double lumen bi-cava cannula for veno-venous extracorporeal membrane oxygenation as bridge to lung transplantation in non-intubated patient. Interact Cardiovasc Thorac Surg (2011).
35. Abrams, D., et al. Insertion of bicaval dual-lumen cannula via the left internal jugular vein for extracorporeal membrane oxygenation. Asaio J 58, 636-637 (2012).
36. Dolch, M. E., et al. Transesophageal echocardiography-guided technique for extracorporeal membrane oxygenation dual-lumen catheter placement. Asaio J 57, 341-343 (2011).
37. Javidfar, J., et al. Use of bicaval dual-lumen catheter for adult venovenous extracorporeal membrane oxygenation. Ann Thorac Surg 91, 1763-1768; discussion 1769 (2011).
38. Wang, D., et al. Wang-Zwische double lumen cannula-toward a percutaneous and ambulatory paracorporeal artificial lung. Asaio j 54, 606-611 (2008).
39. Zhou, X., et al. Long-term support with an ambulatory percutaneous paracorporeal artificial lung. J Heart Lung Transplant 31, 648-654 (2012).
40. Hayes, D., Jr., Kukreja, J., Tobias, J. D., Ballard, H. O. & Hoopes, C. W. Ambulatory venovenous extracorporeal respiratory support as a bridge for cystic fibrosis patients to emergent lung transplantation. J Cyst Fibros 11, 40-45 (2012).
41. Camboni, D., Philipp, A., Mueller, T. & Schmid, C. An important step toward ambulatory veno-venous extracorporeal membrane oxygenation. J Thorac Cardiovasc Surg 144, 282-283; author reply 283-284 (2012).
42. Garcia, J. P., et al. Ambulatory veno-venous extracorporeal membrane oxygenation: innovation and pitfalls. J Thorac Cardiovasc Surg 142, 755-761 (2011).
43. Garcia, J. P., Iacono, A., Kon, Z. N. & Griffith, B. P. Ambulatory extracorporeal membrane oxygenation: a new approach for bridge-to-lung transplantation. J Thorac Cardiovasc Surg 139, e137-139 (2010).
44. Camboni, D., et al. Extracorporeal membrane oxygenation by single-vessel access in adults: advantages and limitations. ASAIO J 58, 616-621 (2012).
45. Turner, D. A., et al. Ambulatory ECMO as a Bridge to Lung Transplant in a Previously Well Pediatric Patient With ARDS. Pediatrics (2014).
46. Hayes, D., Jr., et al. Tracheostomy in adolescent patients bridged to lung transplantation with ambulatory venovenous extracorporeal membrane oxygenation. J Artif Organs 17, 103-105 (2014).
47. Hayes, D., Jr., et al. Pediatric Ambulatory ECMO. Lung (2014).
48. Wang, D., et al. Development of a Double-Lumen Cannula for a Percutaneous RVAD. ASAIO J 61, 397-402 (2015).
49. Condemi, F., et al. Percutaneous Double Lumen Cannula for Right Ventricle Assist Device System: A Computational Fluid Dynamics Study. Biocybern Biomed Eng 36, 482-490 (2016).
50. Kazui, T., et al. Minimally invasive approach for percutaneous CentriMag right ventricular assist device support using a single PROTEKDuo Cannula. Journal of cardiothoracic surgery 11, 123 (2016).

What is claimed is:

1. A multiple lumen device, comprising:
(a) an expandable cage having a proximal end and a distal end;
(b) an outer catheter comprising a first lumen and having a proximal end and a distal end;
(c) an inner catheter comprising a second lumen and a third lumen, and having a proximal end and a distal end, the inner catheter extending through the outer catheter and the expandable cage, and extending beyond the distal end of the expandable cage,
wherein the proximal end of the expandable cage is attached to the outer catheter and the distal end of the expandable cage is attached to the inner catheter, such that when the proximal end of the inner catheter is retracted, the cage moves from a compressed configuration to an expanded configuration.

2. The device of claim 1, and further comprising a pressure monitor in fluid communication with the outer catheter for measuring pressure in the right atrium (RA).

3. The device of claim 1, and further comprising an inflatable balloon at the distal tip of and in fluid communication with the second and third lumens of the inner catheter.

4. The device of claim 3, and further comprising a syringe in fluid communication with the second lumen of the inner catheter for inflating the balloon.

5. The device of claim 3, and further comprising a pressure monitor in fluid communication with the third lumen of the inner catheter for measuring for measuring pressure in the pulmonary artery (PAP) and pulmonary artery wedge pressure (PCWP).

6. The device of claim 1, wherein the wire cage is comprised of super-elastic nitinol wire, stainless steel wire, super elastic polymer, or combinations thereof.

7. The device of claim 1, wherein at least two surgical grade threads are placed around the cage at equal distance from each other.

8. The device of claim 7, wherein the surgical grade threads are comprised of polyester, polypropylene, nylon, or combinations thereof.

9. The device of claim 1, where at least one of the outer catheter or the inner catheter is coated with polytetrafluoroethylene.

10. The device of claim 1, wherein the device is comprised of polyurethane, PVC, silicone, PFTE, polyisoprene, nitrile, or combinations thereof.

11. The device of claim 1, wherein the cage has a maximal expanded diameter from 12 mm to 15 mm.

12. A method for causing blood to flow in a retrograde manner from the pulmonary artery into the right atrium, comprising:
(a) selecting a multiple lumen device comprising:
(i) an expandable cage having a proximal end and a distal end;
(ii) an outer catheter comprising a first lumen and having a proximal end and a distal end;
(iii) an inner catheter comprising a second lumen and a third lumen, and
having a proximal end and a distal end, the inner catheter extending through the outer catheter and the expandable cage, and extending beyond the distal end of the expandable cage,
wherein the proximal end of the expandable cage is attached to the outer catheter and the distal end of the expandable cage is attached to the inner catheter, such that when the proximal end of the inner catheter is retracted, the cage moves from a compressed configuration to an expanded configuration;
(b) percutaneously inserting the multiple lumen device into a blood vessel that leads to the heart;
(c) continuing to insert the multiple lumen device until the distal end of the cage is through the pulmonary valve and into the pulmonary artery;
(d) retracting the inner catheter until the cage is in an expanded configuration;
(e) allowing the desired amount of blood to flow in a retrograde manner from the pulmonary artery into the right atrium;
(f) extending the inner catheter until the cage is in a compressed configuration; and
(g) removing the multiple lumen device from the blood vessel.

13. A multiple lumen device, comprising:
(a) an expandable cage having a proximal end and a distal end;
(b) an outer catheter comprising a first lumen and having a proximal end and a distal end;
(c) an inner catheter comprising a second lumen and a third lumen, and having a proximal end and a distal end, the inner catheter extending through the outer catheter and the expandable cage, and extending beyond the distal end of the expandable cage,
wherein at least one of the proximal end and the distal end of the expandable cage is attached to the inner catheter, such that when the proximal end of the inner catheter is retracted, the cage is collapsed within the outer catheter into a compressed configuration and when the proximal end of the outer catheter is retracted, the cage extends from the distal end of the outer catheter into an expanded configuration.

14. The device of claim 13, wherein both the proximal end and the distal end of the expandable cage are attached to the inner catheter.

15. The device of claim 13, wherein only one of the proximal end and the distal end of the expandable cage are attached to the inner catheter.

16. The device of claim 13, and further comprising a pressure monitor in fluid communication with the outer catheter for measuring pressure in the right atrium (RA).

17. The device of claim 13, and further comprising an inflatable balloon at the distal tip of and in fluid communication with at least one of the second and third lumens of the inner catheter.

18. The device of claim 17, and further comprising a syringe in fluid communication with the second lumen of the inner catheter for inflating the balloon.

19. The device of claim 17, and further comprising a pressure monitor in fluid communication with the third lumen of the inner catheter for measuring for measuring pressure in the pulmonary artery (PAP) and pulmonary artery wedge pressure (PCWP).

20. The device of claim 13, wherein the cage has a maximal expanded diameter from 12 mm to 15 mm.
